(19) United States
(12) Patent Application Publication
(10) Pub. No.: US 2024/0000331 A1
(43) Pub. Date: Jan. 4, 2024

(54) INTRACRANIAL PRESSURE SENSOR

(71) Applicant: KOC UNIVERSITESI, (US)

(72) Inventors: Levent BEKER, Istanbul (TR); Rahul SINGH, Istanbul (TR)

(73) Assignee: KOC UNIVERSITESI, Istanbul (TR)

(21) Appl. No.: 18/035,147

(22) PCT Filed: Nov. 3, 2021
(86) PCT No.: PCT/TR2021/051130

§ 371 Date: May 3, 2023

(30) Foreign Application Priority Data
Nov. 3, 2020 (TR) 2020/17548

(51) Int. Cl. A61B 5/03; A61B 5/00

(52) U.S. Cl. CPC A61B 5/031; A61B 5/6868; A61B 2562/0247; A61B 2562/12

(57) ABSTRACT

A novel bioresorbable intracranial pressure sensor fabricated using readily available bioresorbable materials which are compatible with standard microfabrication technology is provided. The intracranial pressure sensor can also stay operational for a relevant time scale and can transfer data wirelessly.

### CROSS REFERENCE TO RELATED APPLICATIONS

This application is the national stage entry of International Application No. PCT/TR2021/051130 filed Nov. 3, 2021, and which is based upon and claims priority to Turkish Patent Application No. 2020/17548 filed Nov. 3, 2020, the entire contents of which are incorporated by reference herein.

### FIELD OF INVENTION

The present invention relates to an implantable medical device, especially to an intracranial pressure sensor for measuring intracranial pressure.

### BACKGROUND

Post-operative monitoring of intracranial pressure (ICP) can help prevent adverse effects of elevated ICP in the case of traumatic brain injury and shunt implantation for hydrocephalus.

Conventional implantable medical devices (IMDs) are made up of non-resorbable materials that can lead to adverse immune responses such as chronic inflammation at the implant location, or even risk of infections. Therefore, the IMDs should be renewed periodically or they should be removed from the body altogether. For removing the IMDs, extraction surgeries are often required, which eventually results in an additional cost of the treatment and patient discomfort. In addition, restrictions are imposed on the patient's movement due to the wires connecting the implantable device to an external reader. The wires protruding out of the skin are also a potential source of epidermal infections.

Therefore, a need exists in the field for novel 1 MB which does not require an extraction surgery in order to be removed from the body. Additionally, an IMD which reduces the risk of infection is also needed.

Various forms of silicon, such as silicon nano membrane, silicon nano ribbons, silicon oxides/nitrides and porous silicon, have been known for their bioresorbable properties. Some studies have utilized piezoresistive properties of silicon nano membrane for measurement of pressure. However, fabrication of such silicon nanomembrane sensors require a unique transfer printing technology which can be achieved only through a complex, custom-built, and expensive piece of equipment. Another drawback of the piezoresistive pressure sensor is difficulty in coupling with wireless data transfer network. Wireless communication at a reasonable distance between the implanted sensor and outside world is essential for not only improving the practicality of the implanted sensor but also a substantial reduction in the risk of infection since there are no wires or connection lines through the skin. Similarly, an optical pressure sensor also faces the risk of infection due to optical fiber passing through the skin.

Most of the bioresorbable MEMS (Micro Electro-Mechanical Systems) pressure sensors are based on piezoresistivity, which must be connected to a power source such as a bioresorbable battery for continuous operation. On the other hand, capacitive sensors can be coupled with an inductor or antenna for wireless, battery-free operation. A typical capacitive pressure sensor consists of a parallel plate scheme, that is, a deformable diaphragm acting as a top electrode and a bottom electrode on the substrate. These double-plate capacitive pressure sensors can be designed to be highly sensitive; however, the major drawback is the onset of the dissolution of the top electrode immediately after the implantation. This leads to implant failure, which has not been an issue with conventional non-resorbable sensors.

Therefore, a need exists in the field for novel bioresorbable pressure sensor fabricated using readily available bioresorbable materials which are compatible with standard microfabrication technology. A further need exists for a bioresorbable pressure sensor that can stay operational for a relevant time scale and can transfer data wirelessly.

The European patent application numbered EP2547258 discloses an implantable biomedical device on a bioresorbable substrate. Said biomedical device does not disclose sealing of the electrodes and hence, the onset of the dissolution of the top electrode is initiated immediately after the implantation.

### SUMMARY

The objective of the invention is to provide an intracranial pressure sensor for measuring the intracranial pressure.

Another objective of the invention is to provide a bioresorbable intracranial pressure sensor.

Yet another objective of the invention is to provide a bioresorbable intracranial pressure sensor whose electrodes do not start dissolving immediately after the implantation.

Yet another objective of the invention is to provide a bioresorbable intracranial pressure sensor capable of providing data wirelessly.

### BRIEF DESCRIPTION OF THE DRAWINGS

The intracranial pressure sensor in order to fulfill the objects of the present invention is illustrated in the attached figures, where:

FIG. 1 shows a cross-sectional view of the doped silicon substrate with insulating layer.

FIG. 2 shows a cross-sectional view of the doped silicon substrate with the insulating layer and coating of photoresist.

FIG. 3 shows a cross-sectional view of the doped silicon substrate after photolithography and development.

FIG. 4 shows a cross-sectional view of the doped silicon substrate after deposition of conductive layer and lift off.

FIG. 5 shows a cross-sectional view after deposition of the second insulating layer.

FIG. 6 shows a cross-sectional view after photoresist coating and patterning.

FIG. 7 shows a cross-sectional view after selective etching and photoresist strip.

FIG. 8 shows a cross-sectional view after deposition of the sacrificial layer.

FIG. 9 shows a cross-sectional view after patterning and selective etching of the sacrificial layer.

FIG. 10 shows a cross-sectional view after deposition of the structural layer.

FIG. 11 shows a cross-sectional view after patterning and selective etching of the structural layer.

FIGS. 12A-12 show schematic of the structures after removal of the sacrificial layer. FIG. 12A shows a side view of the structures with deformable diaphragm and openings for the electrical connections. And FIG. 12B shows a top view of the structure with diaphragm and anchor points.

FIG. 13 shows a cross-sectional view after deposition of the sealing layer on the diaphragm.

FIG. 14 shows a cross-sectional view after patterning and selective etching of the sealing layer.

FIGS. 15A-15B show schematics of the first embodiment of the sensor. FIG. 15A shows a side of the first embodiment of the sensor after sealing of the cavity and deposition of metal connection lines. And FIG. 15B shows a op view of the first embodiment of the sensor with a sealed cavity and metal connection points.

FIG. 16 shows a cross-sectional view of the intracranial pressure sensor after converting selected regions of the doped silicon substrate into porous silicon.

FIG. 17 shows a schematic of planar interdigitated electrodes.

FIG. 18 shows a comparison between interdigitated and parallel plate capacitive sensor.

### DETAILED DESCRIPTION OF THE INVENTION

Elements shown in the FIGS. are individually numbered, and the correspondence of these numbers are given as follows:
1. Intracranial pressure sensor
100. Substrate
102. Insulating layer
104. Photoresist layer
106. Electrodes
108. Second insulating layer
110. Sacrificial layer
112. Structural layer
114. Anchor
116. Cavity
118. Sealing Layer
120. Interconnects
122. Porous silicon form
124. First terminal
126. Second terminal
128. Electrodes gap
202. Recess
204. Gap
206. Etch window
208. Etching location
210. Anchor opening
212. Connection opening

Referring to FIG. 16, the intracranial pressure sensor (1) comprises;
at least one bioresorbable porous silicon form (122),
at least one substrate (100) which is either converted to bioresorbable porous silicon form (122) or comprises other rigid bioresorbable materials, such as ZnO ceramic
at least one insulating layer (102) for providing electrical isolation, formed on the substrate (100)
at least one second insulating layer (108) containing at least two electrodes (106), having at least two openings for exposing the said electrodes (106),
at least one structural layer (112) connected to the second insulating layer (108) via at least one anchor (114) such that it forms at least one cavity (116) between the structural layer (112) and the second insulating layer (108),
at least one sealing layer (118) for sealing the cavity (116) and
at least two interconnects (120) for providing means for electrical connection to the electrodes (106).

Referring again to FIG. 16, he intracranial pressure sensor (1) comprises at least one bioresorbable porous silicon form (122) and at least one substrate (100) located on the said porous silicon form (122). The substrate (100) is preferably doped. The doping material is preferably a p-type material such as Boron or an n-type material such as Phosphorous.

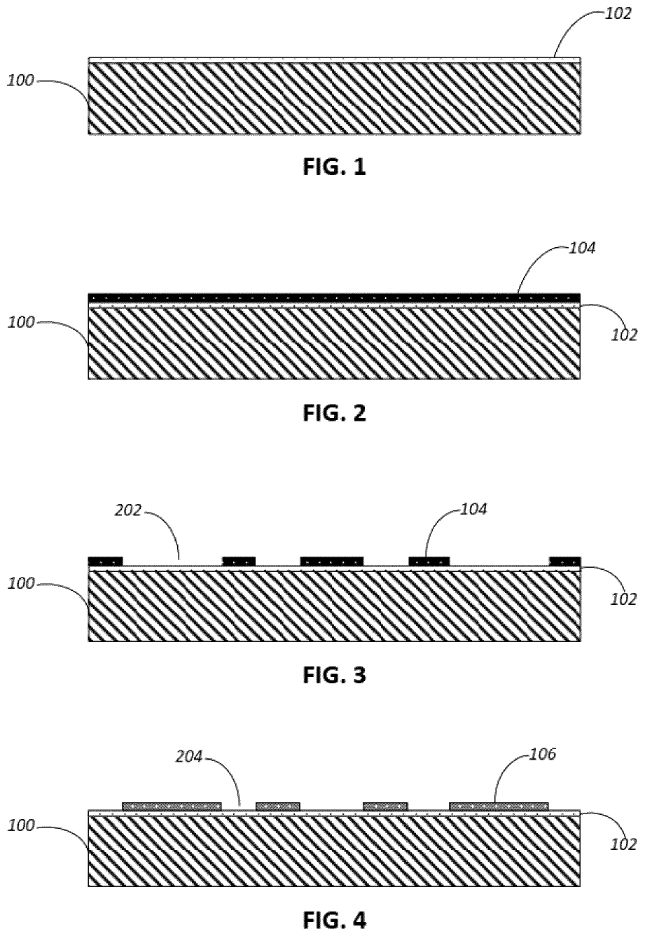

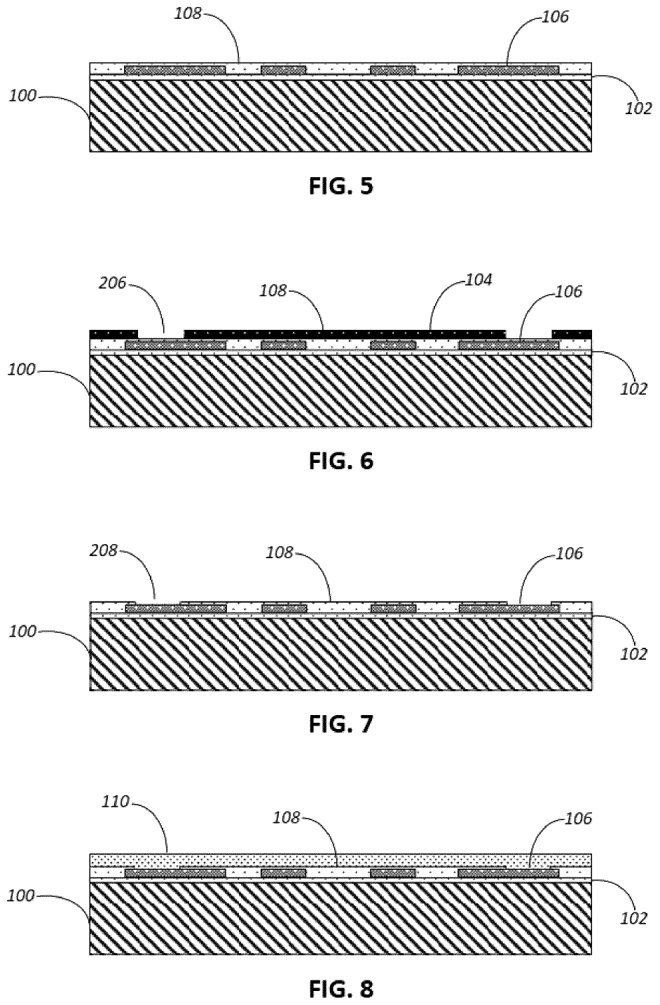

As shown in FIGS. 1-12A, 13-15A, and 16, on the substrate (100), at least one insulating layer (102) for providing electrical isolation, is formed. The insulating layer (102) may be of SiO₂ or Si₃N₄ or both. It might be deposited through standard physical or chemical vapor deposition processes. As shown in FIGS. 4-12A, 13-15A, and 16, the insulating layer (102) provides electrical isolation between the electrodes (106) and the substrate (100).

Referring back to FIG. 16, the inventive intracranial pressure sensor (1) comprises at least two electrodes (106) which are interdigitated with each other. Said electrodes (106) might be formed by bioresorbable metals such as magnesium (Mg), molybdenum (Mo), zinc (Zn), Tungsten (W) or iron (Fe) or compounds and/or alloys thereof.

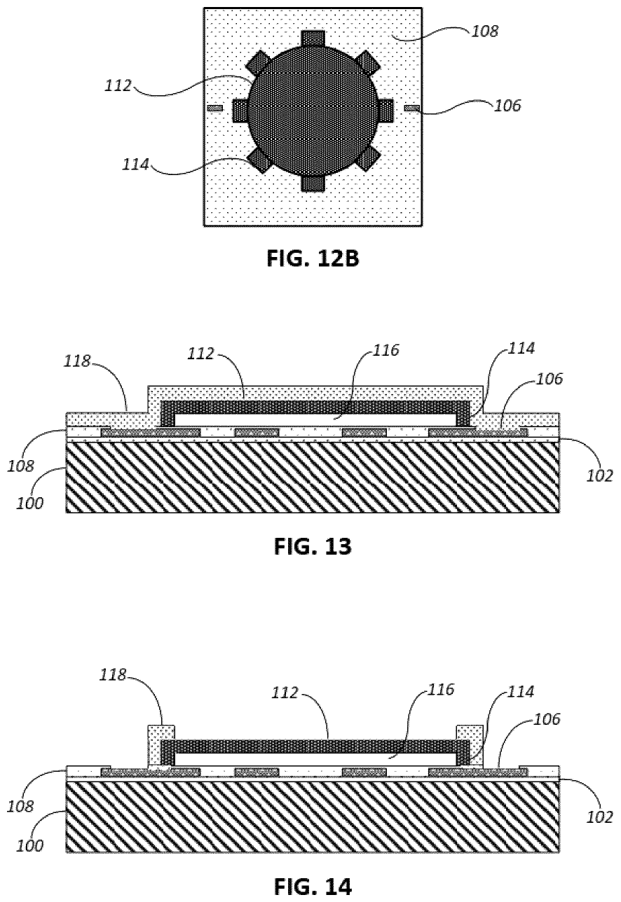

As shown in FIG. 12A, the electrodes (106) are covered by at least one second insulating layer (108). The second insulating layer (108) has at least two connection openings (212) for exposing at least a part of the said electrodes (106), at least one connection opening (212) for each electrode (106). By the virtue of these connection openings (212), an electrical connection between the electrodes (106) and other components such as an inductor or antenna is made possible.

As shown in FIGS. 11-16, at least one structural layer (112) is connected to the second insulating layer (108) via at least one anchor (114) such that it forms at least one cavity (116) between the structural layer (112) and the second insulating layer (108). The structural layer (112) might be deposited through standard physical or chemical vapor deposition processes. The structural layer (112) acts as a deformable diaphragm and fabricated, preferably, from a silicon based inorganic material, including but not limited to polycrystalline silicon (poly Si), amorphous silicon (aSi) or silicon oxide/nitride (SiO₂/Si₃N₄) or a combination thereof. The thickness of the structural layer is preferably between 1 μm-3 μm.

As shown in FIGS. 13-16, the cavity (116) is sealed by at least one sealing layer (118). The thickness of the sealing layer (118) is preferably between 1 μm-3 μm. The sealing layer (118) material is preferably silicon dioxide (SiO₂), however it could also be a different material such as silicon nitride (Si₃N₄).

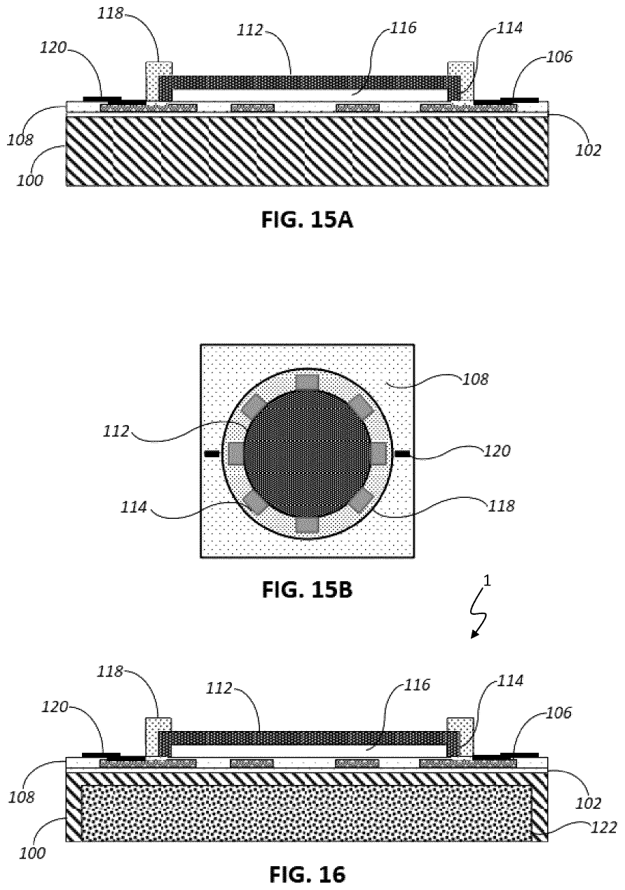

As shown in FIGS. 15A-16, at least two interconnects (120) are deposited on the exposed parts of the electrodes (106). The interconnects (120) might be deposited through standard physical or chemical vapor deposition processes. The interconnects (120) provide means for electrical connection to the electrodes (106).

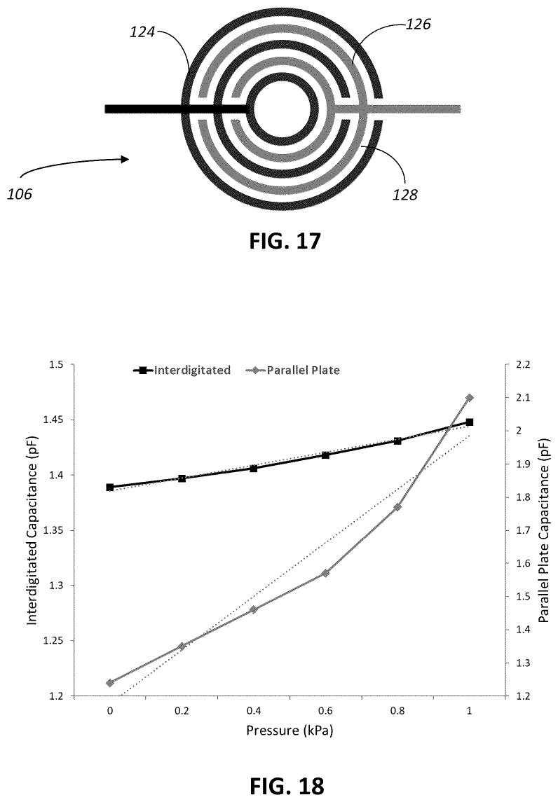

The interdigitated electrodes (106), shown in FIG. 17, are sealed under the deformable structural layer (112) (diaphragm). The capacitance in this configuration depends on the electric field in the close proximity of the electrodes (106), which will be highly dependent on the diaphragm's deformation. Thus, intracranial pressure value can be obtained with respect to change in capacitance. Since the electrodes (106) do not come in contact with surrounding biofluids, the sensor can stay functioning for a longer period. Furthermore, by coating the structural layer (112) or the whole device using a triggered bioresorbable material, the degradation can be initiated at the desired time. The said triggered bioresorbable material may be an inorganic material such as SiO₂ or Si₃N₄, a natural polymer such as silk or wax, or synthetic polymers such as PLGA, PCL or polyanhydrides. However, said triggered bioresorbable materials are not limited to the above-mentioned examples. By providing a trigger, the degradation can be initiated. Said trigger may be in the form of heat, radiation such as UV or infrared radiation, or some kind of solvent. However, the said trigger is not limited to the above-mentioned examples. After a specific trigger is provided, said coating layer starts to dissolve, for example, wax coating will melt when heat is generated. Hence, exposing the device to body fluids

In an alternative embodiment of the invention, the inventive intracranial pressure sensor (1) comprises an antenna (not shown). When connected to an antenna, the capacitance changes (ΔC) of the electrodes (106) result in a shift in the resonance frequency (Δf₀) of the inductor-capacitor (LC) system. Here, the inductor is the antenna and the capacitor is the electrodes (106). This frequency shift is given by Δf₀=−ΔC4π(LC)^{-1/2}, which can be detected through an RF reader system and hence intracranial pressure can be monitored. Therefore, such a design can have required resolution (±1 mmHg) and can operate for a considerably longer period with wireless data transmission capability.

A novel production method for the above-mentioned intracranial pressure sensor (1) is as follows:

A doped silicon substrate (100) and a layer of insulating layer (102) is provided (FIG. 1).

A photoresist layer (104) is applied on the insulating layer (102) (FIG. 2). The photoresist layer (104) is preferably coated on the insulating layer (102) using spin coating method.

Afterwards, the photoresist layer (104) is patterned, preferably using photolithography, and then developed in a developer solution. After development, specific parts of photoresist (104), i.e. the parts defined with the above-mentioned patterning step, are removed, thereby forming recesses (202). The photoresist layer (104) acts as a mask for the formation of electrodes (106) (FIG. 3).

The electrodes (106) are formed in the said recesses (202) by deposition of bioresorbable metals. An exemplary set of these metals are mentioned in the previous parts of the description. However, any bioresorbable metal, compounds and/or alloys thereof are usable. Afterwards, the remaining photoresist layer (104) is removed, and gaps (204) are formed between the interdigitated electrodes (106) (FIG. 4).

A second insulating layer (108) is deposited on the electrodes preferably through physical or chemical vapor deposition processes (FIG. 5).

A photoresist layer (104) is deposited on the second insulating layer (108), preferably through spin coating method. The said photoresist layer (104) is then patterned using photolithography. Photoresist layer (104) is removed at specific locations using developer solution to form etch windows (206) (FIG. 6). The second insulating layer (108) is then etched at said locations to expose a section of both electrodes (106) (FIG. 7). The exposed sections of the electrodes (106) are named openings (208). The openings (208) act as connection points to couple the electrodes with other elements of the sensor (1) such as an inductor or antenna.

Afterwards, a sacrificial layer (110) is formed on the second insulating layer (108), preferably through standard physical or chemical vapor deposition processes (FIG. 8). The thickness of the sacrificial layer (110) depends on the desired gap between the diaphragm and the electrodes which may vary from 0.31 μm-21 μm. The material of the sacrificial layer (110) also depends on the diaphragm material. For instance, if the diaphragm material is amorphous silicon, then silicon dioxide (SiO₂) may be used as sacrificial layer (110) or vice versa. The material of the diaphragm (i.e. the structural layer (112)) should be different than the material of the sacrificial layer (110).

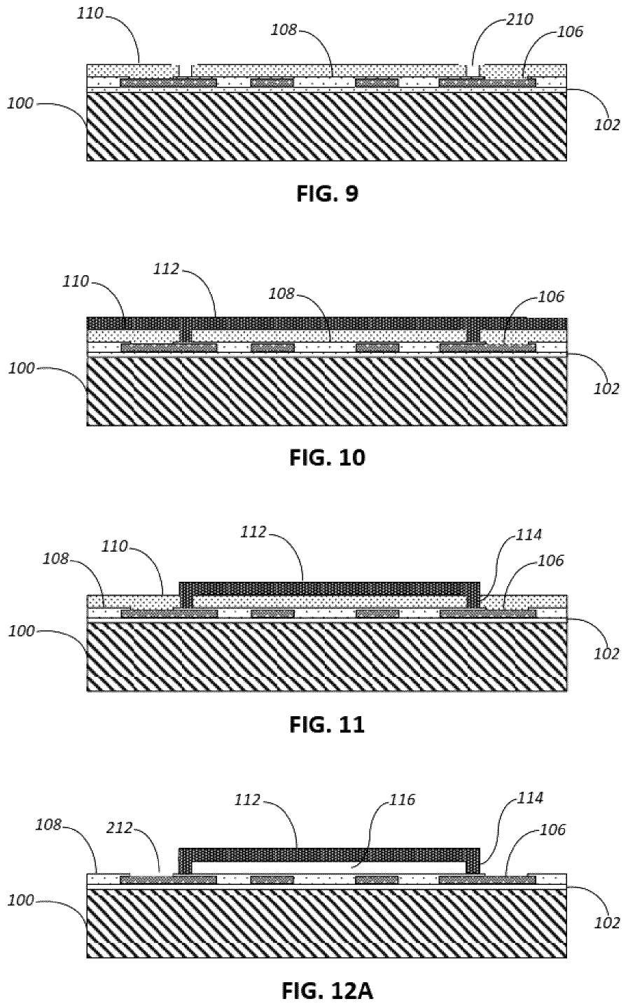

Afterwards, the sacrificial layer (110) is selectively etched, preferably using photolithography, to form anchor openings (210) for diaphragm anchors (FIG. 9).

A structural layer (112) is deposited on the sacrificial layer (110) and inside the anchor openings (210) (FIG. 10). The structural layer (112) acts as a deformable diaphragm. The structural layer (112) is preferably deposited through standard physical or chemical vapor deposition process. The structural layer (112) is fabricated preferably using silicon based inorganic materials such as polycrystalline silicon (poly Si), amorphous silicon (aSi) or silicon oxide/nitride (SiO₂/Si₃N₄). The thickness of the structural layer (112) may vary preferably from 1 μm-3 μm.

The structural layer (112) is then selectively etched using photolithography, and thus, the anchors (114) are formed (FIG. 11). The parts of the structural layer (112) that are removed are preferably the parts outwards the anchor openings (210).

The sacrificial layer (110) is then removed by wet etching process. This process releases the diaphragm, forms a cavity (116) between the diaphragm and the electrodes (106), and forms the openings (212) for connection with other sensor elements such as an inductor and antenna (FIGS. 12A and 12B).

Afterwards, a sealing layer (118) is deposited preferably through standard physical or chemical vapor deposition processes (FIG. 13). The sealing layer (118) material may be silicon oxide (SiO₂). The thickness of the sealing layer (112) may vary preferably from 1 μm-3 μm.

Afterwards, the sealing layer (118) is selectively removed through photolithography and wet etching process (FIG. 14). The parts of the sealing layer (118) on top of the openings (212) and on top of the structural layer (112) are preferably removed, thereby leaving the parts next to the anchors. This enables sealing the anchors, while not preventing the structural layer (112) from deforming under the effect of pressure.

Afterwards, metal interconnects (120) are deposited on exposed electrodes (106) through preferably physical vapor deposition process. (FIGS. 15A, 15B).

The doped silicon substrate (100) is then selectively converted into bioresorbable porous silicon form (122) through electrochemical etching process in an electrochemical etch cell.

Forming of the porous silicon form (122) in the final step eliminates the need of fabricating the bioresorbable substrate and the electronic components separately and utilizing custom equipment to bond them together due to highly brittle nature of porous silicon layers.

In an exemplary embodiment, the interdigitated electrodes (106) geometry consist of first terminal (124) and the second terminal (126) in circular form with gap (128) between the electrodes (FIG. 17). The pressure response of the sensor can be tuned by varying the number of turns in the electrodes, width of the electrodes and gap between the electrodes.

The response of an interdigitated sensor (30 electrodes) is compared with a parallel plate sensor using finite element analysis (FIG. 18). The diameter of the sensors in both cases is 300 μm. Results show that the nominal capacitance and linearity of the sensor response of the interdigitated sensor higher compared to the parallel plate sensor for the given pressure range. This characteristic enables an interdigitated sensor to be used for wider pressure range compared to a parallel plate capacitive sensor

What is claimed is:

1. An intracranial pressure sensor for measuring intracranial pressure, comprising;
at least one bioresorbable porous silicon form,
at least one substrate which is either converted to bioresorbable porous silicon form or comprises other rigid bioresorbable materials, such as ZnO ceramic,
at least one insulating layer for providing electrical isolation, formed on the substrate,
at least one second insulating layer containing at least two electrodes, having at least two openings for exposing the said electrodes,
at least one deformable structural layer connected to the second insulating layer via at least one anchor such that it forms at least one cavity between the structural layer and the second insulating layer,
at least one sealing layer for sealing the cavity and
at least two interconnects for providing means for electrical connection to the electrodes.

2. The intracranial pressure sensor according to claim 1 wherein the substrate is doped.

3. The intracranial pressure sensor according to claim 1, wherein the at least one insulating layer is made of SiO₂ or Si₃N₄ or both.

4. The intracranial pressure sensor according to claim 1, wherein the at least one insulating layer is deposited through standard physical or chemical vapor deposition processes.

5. The intracranial pressure sensor according to claim 1, wherein the at least two electrodes are interdigitated with each other.

6. The intracranial pressure sensor according to claim 1, wherein the electrodes are formed by bioresorbable metals such as magnesium (Mg), molybdenum (Mo), zinc (Zn), Tungsten (W) or iron (Fe) or compounds and/or alloys thereof.

7. The intracranial pressure sensor according to claim 1, wherein the structural layer is deposited through standard physical or chemical vapor deposition processes.

8. The intracranial pressure sensor according to claim 1, wherein the structural layer is fabricated from a silicon based inorganic material, including but not limited to polycrystalline silicon (poly Si), amorphous silicon (aSi) or silicon oxide/nitride (SiO₂/Si₃N₄) or a combination thereof.

9. The intracranial pressure sensor according to claim 1, wherein the thickness of the structural layer is between 1 μm-3 μm.

10. The intracranial pressure sensor according to claim 1, wherein the thickness of the sealing layer is between 1 μm-3 μm.

11. The intracranial pressure sensor according to claim 1, wherein the sealing layer material is made of silicon dioxide (SiO₂) or any other bioresorbable material.

12. The intracranial pressure sensor according to claim 1, wherein the at least two interconnects are deposited on the exposed parts of the electrodes.

13. The intracranial pressure sensor according to claim 1, wherein the structural layer is coated with a triggered bioresorbable material.

14. The intracranial pressure sensor according to claim 1, wherein the said intracranial pressure sensor further comprises an antenna that is electrically connected to the interconnects.

15. A method for producing the intracranial pressure sensor according to claim 1 wherein the method comprises the following steps:
providing a doped silicon substrate and a layer of insulating layer
applying a photoresist layer on the insulating layer
patterning and developing the photoresist layer, and removing the specific parts of the photoresist that are defined by the patterns formed, thereby forming recesses,
forming the electrodes in the said recesses by deposition of bioresorbable metals, and removing the remaining photoresist layer, thereby forming gaps between the interdigitated electrodes,
depositing a second insulating layer on the electrodes,
depositing a photoresist layer on the second insulating layer,
patterning the said photoresist layer using photolithography.
removing the photoresist layer at specific locations using developer solution to form etch windows and etching the second insulating layer at said locations, thereby exposing a section of both electrodes,
forming a sacrificial layer on the second insulating layer,
Selectively etching the sacrificial layer, thereby forming anchor openings for diaphragm anchors,
depositing a structural layer of a material different than the material of the sacrificial layer, on the sacrificial layer and inside the anchor openings,
selectively etching the structural layer, thereby forming the anchors,
removing the sacrificial layer by wet etching process,
depositing a sealing layer,
selectively removing the sealing layer through photolithography and wet etching processes,
depositing metal interconnects are deposited on exposed electrodes, and
selectively converting the doped silicon substrate into bioresorbable porous silicon form (122) through electrochemical etching process in an electrochemical etch cell.

16. The method according to claim 15 wherein the photoresist layer is coated on the insulating layer using spin coating method.
